(19) United States
(12) Patent Application Publication
(10) Pub. No.: US 2007/0043091 A1
(43) Pub. Date: Feb. 22, 2007

(54) Kv1.5-BLOCKER FOR THE SELECTIVE INCREASE OF ATRIAL CONTRACTILITY AND TREATMENT OF CARDIAC INSUFFICIENCY

(75) Inventor: Klaus WIRTH, Kriftel (DE)

Correspondence Address: ROSS J. OEHLER;SANOFI-AVENTIS U.S. LLC, 1041 ROUTE 202-206, MAIL CODE: D303A, BRIDGEWATER, NJ, 08807, US

(73) Assignee: SANOFI-AVENTIS DEUTSCHLAND GMBH, Frankfurt am Main (DE)

(21) Appl. No.: 11/466,791

(22) Filed: Aug. 24, 2006

Related U.S. Application Data
(63) Continuation of application No. PCT/EP05/01422, filed on Feb. 12, 2005.

(30) Foreign Application Priority Data
Feb. 26, 2004 (DE) 102004009931.6

(51) Int. Cl. A61K 31/44

(52) U.S. Cl. 514/357; 514/352

(57) ABSTRACT

The invention relates to the atrial contractility-increasing effect of Kv1.5 blockers, especially phenylcarboxamides of the formulae Ia or Ib 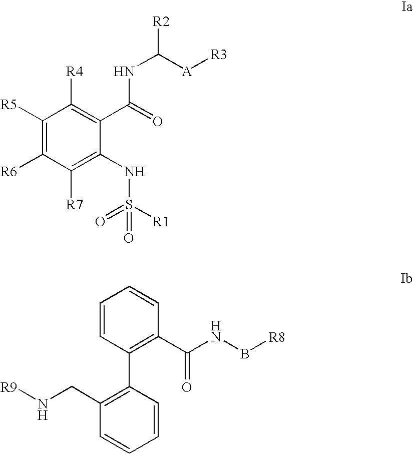 or pharmaceutically acceptable salts thereof, for treating reduced atrial contractility and heart failure, especially heart failure caused by diastolic dysfunction.

### CROSS-REFERENCE TO RELATED APPLICATIONS

This application is a continuation of PCT/EP2005/001422, fied Feb. 12, 2005, and claims the benefit of U.S. Provisional Application No. 60/591,649, filed Jul. 28, 2004, and German Application No.10-2004-009931, filed Feb. 26, 2004, all incorporated herein by reference.

### FIELD OF THE INVENTION

The invention relates to the atrial contractility-increasing effect of Kv1.5 blockers, especially phenylcarboxamides of the formulae Ia and/or Ib 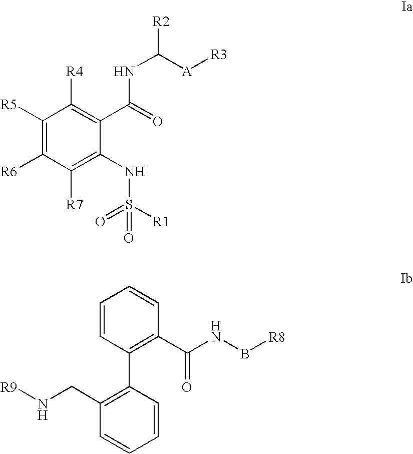
and/or pharmaceutically acceptable salts thereof, for treating reduced atrial contractility and heart failure, especially heart failure caused by diastolic dysfunction.

### BACKGROUND OF THE INVENTION

Atrial fibrillation (AF) and atrial flutter are the commonest persistent cardiac arrhythmias. The incidence increases with increasing age and frequently leads to fatal sequelae such as, for example, stroke. AF affects about 1 million Americans each year and leads to more than 80 000 strokes each year in the USA. In the elderly, and as a consequence of atrial fibrillation, there is impairment of atrial contraction, which is referred to as atrial stunning. In such cases there is weakening of active atrial contraction, enlargement of the atria and decreased filling of the ventricles. The reduced ventricular filling leads to decreased ejection from the heart and thus decreased physical exercise tolerance.

The deterioration in atrial function has overall hemodynamic, prothrombotic and arrhythmogenic effects. It impairs the performance of the heart, especially during physical exercise. The deficient atrial contractility may lead to stagnation of blood in the atrium, causing thrombus formation and subsequent embolisms (stroke). Atrial stunning leads to dilatation of the atrium, which considerably increases the tendency of the atrium to arrhythmias. A decrease in the size of the atrium by increasing its contractility therefore reduces the susceptibility to arrhythmias and thus offers protection against reinitiation of atrial fibrillation.

Apart from the benefits of increasing the atrial contractility for the atrium itself, a selective increase in atrial contractility is therapeutically beneficial in the treatment of heart failure, especially when it is based on diastolic dysfunction. This is because in such a case there is impairment of filling of the left ventricle which is based on a decreased extensibility and elasticity of the ventricle. Such an impairment is frequently associated with cardiac hypertrophy or cardiomyopathies, where the walls of the heart may be thickened or fibrosed. The impaired extensibility is also referred to as decreased ventricular compliance. This term implies that the extensibility of the ventricle is in principle retained but adequate extension and thus filling of the ventricles can be achieved only by applying a greater force (higher filling pressure). Active atrial contraction generates the necessary filling pressure for the ventricle. It is possible by increasing atrial contractility beyond the normal level to improve the impaired ventricular function. Positive inotropic substances such as cardiac glycosides are unsuitable for this because, in particular, they increase ventricular contraction directly and thus reduce the size of the ventricle, so that filling of the ventricle now deteriorates again despite a possible simultaneous contractility-increasing effect on the atria. A selective increase in atrial contractility is necessary for this.

It has been found in experiments on anesthetized pigs that Kv1.5 blockers selectively increase atrial contractility without directly influencing ventricular contractility. It was likewise possible to show in pigs that the atrial contractility-increasing effect leads to an improved circulatory situation when filling of the ventricle is impeded experimentally (model of diastolic dysfunction). A significant improvement in the reduced cardiac output, the crucial parameter of the performance of the heart, was possible with Kv1.5 blockers. These experiments demonstrate the selective atrial increase in contractility by Kv1.5 blockers and their beneficial effects on heart failure, especially diastolic heart failure.

### SUMMARY OF THE INVENTION

The invention relates to the use of a compound of the formula Ia or Ib 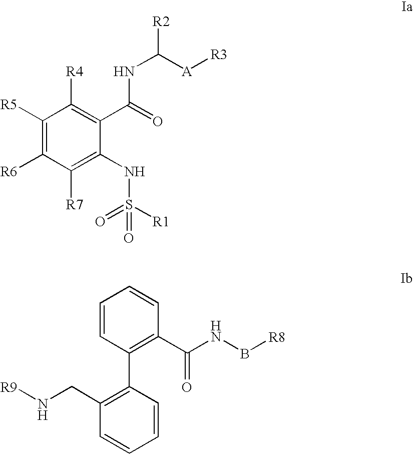
or a physiologically tolerated salt thereof for the treatment or prophylaxis of heart failure,

wherein the meanings are

R1 is alkyl having 3, 4 or 5 C atoms or quinolinyl,

R2 is alkyl having 1, 2, 3 or 4 C atoms or cyclopropyl;

R3 is phenyl or pyridyl,
where the phenyl or pyridyl is unsubstituted or substituted by 1 or 2 substituents selected from the group consisting of F, Cl, CF₃, OCF₃, alkyl having 1, 2 or 3 C atoms and alkoxy having 1, 2 or 3 C atoms;

A is —CₙH₂ₙ—;
n is 0, 1 or 2;

R4, R5, R6 and R7
are independently of one another hydrogen, F, Cl, CF₃, OCF₃, CN, alkyl having 1, 2 or 3 carbon atoms, or alkoxy having 1, 2 or 3 carbon atoms;

B is —CₘH₂ₘ—;
m is 1 or 2;

R8 is alkyl having 2 or 3 carbon atoms, phenyl or pyridyl, where the phenyl or pyridyl is unsubstituted or substituted by 1 or 2 substituents selected from the group consisting of F, Cl, CF₃,
OCF₃, alkyl having 1, 2 or 3 carbon atoms and alkoxy having 1, 2 or 3 carbon atoms;

R9 is C(O)OR10 or COR10;

R10 is —CₓH₂ₓ—R11;
x is 0, 1 or 2;
R11 is phenyl,
where the phenyl is unsubstituted or substituted by 1 or 2 substituents selected from the group consisting of F, Cl, CF₃,
OCF₃, alkyl having 1, 2 or 3 carbon atoms and alkoxy having 1, 2 or 3 carbon atoms.

One embodiment of the invention is directed to the use of a compound of the formulae Ia or Ib selected from the group consisting of
N-(2-pyridin-3-ylethyl)-2′-{[2-(4-methoxyphenyl)acetylamino]methyl}bi-phenyl-2-carboxamide,
N-(2-(2-pyridyl)ethyl)-2′-(benzyloxycarbonylaminomethyl)biphenyl-2-carboxamide,
N-(2,4-difluorobenzyl)-2′-{[2-(4-methoxyphenyl)acetylamino]methyl}-biphenyl-2-carboxamide,
N-(2-(2-pyridyl)ethyl)-(S)-2′-(α-methylbenzyloxycarbonylaminomethyl)bi-phenyl-2-carboxamide,
2-(butyl-1-sulfonylamino)-N-[1(R)-(6-methoxypyridin-3-yl)propyl]benzamide,
2-(butyl-1-sulfonylamino)-N-(cyclopropylpyridin-3-ylmethyl)-5-methyl-benzamide,
(S)-5-fluoro-2-(quinoline-8-sulfonylamino)-N-(1-phenylpropyl)benzamide and the physiologically tolerated salts thereof.

A particular embodiment of the invention is directed to the use of a compound of the formula Ia or Ib or a physiologically tolerated salt thereof for the treatment or prophylaxis of diastolic heart failure.

### DETAILED DESCRIPTION OF THE INVENTION

Alkyl radicals and alkylene radicals may be straight-chain or branched. This also applies to the alkylene radicals of the formulae CₙH₂ₙ, CₘH₂ₘ and CₓH₂ₓ. Alkyl radicals and alkylene radicals may also be straight-chain or branched if they are substituted or present in other radicals, for example in an alkoxy radical. Examples of alkyl radicals are methyl, ethyl, n-propyl, isopropyl, n-butyl, isobutyl, sec-butyl, tert-butyl or n-pentyl. The divalent radicals derived from these radicals, for example methylene, 1,1-ethylene, 1,2-ethylene, 1,1-propylene, 1,2-propylene, etc., are examples of alkylene radicals.

Pyridyl is either 2-, 3- or 4-pyridyl.

Quinolinyl includes 2-, 3-, 4-, 5-, 6-, 7- or 8-quinolyl, with preference for the 8-quinolyl radical.

Monosubstituted phenyl radicals may be substituted in the 2, 3 or 4 position, and disubstituted in the 2,3, 2,4, 2,5, 2,6, 3,4 or 3,5 position. Corresponding statements also apply analogously to the pyridyl radicals.

If a radical is disubstituted, the substituents may be identical or different.

If the compounds of the formula Ia or Ib contain one or more acidic or basic groups or one or more basic heterocycles, the invention also includes the corresponding physiologically or toxicologically acceptable salts, especially the pharmaceutically usable salts. Thus, the compounds of the formula la may be deprotonated on the sulfonamide group and be used for example as alkali metal salts, preferably sodium or potassium salts, or as ammonium salts, for example as salts with ammonia or organic amines or amino acids. Compounds of the formula Ia or Ib comprising a pyridine or quinoline substituent may also be used in the form of their physiologically tolerated acid addition salts with inorganic or organic acids, for example as hydrochlorides, phosphates, sulfates, methanesulfonates, acetates, lactates, maleates, fumarates, malates, gluconates, etc.

The compounds of the formula Ia or Ib may, when appropriately substituted, exist in stereoisomeric forms. If the compounds of the formula Ia or Ib contain one or more centers of asymmetry, these may have independently of one another the S configuration or the R configuration. The invention includes all possible stereoisomers, for example enantiomers or diastereomers, and mixtures of two or more stereoisomeric forms, for example enantiomers and/or diastereomers, in any ratios. The invention thus includes for example enantiomers in enantiomer pure form, both as levorotatory and as dextrorotatory antipodes, and in the form of mixtures of the two enantiomers in various ratios or in the form of racemates. Individual stereoisomers can be prepared as desired by fractionating a mixture by conventional methods or for example by using isomerically pure synthons.

The compounds of the formulae Ia or Ib can be prepared in accordance with the preparation methods described in WO 0125189, WO 02088073 or WO 02100825.

The compounds of the formulae Ia or Ib can be employed on their own, mixed with one another or in the form of pharmaceutical preparations in humans or animals according to the invention for the treatment of heart failure.

Pharmaceutical preparations comprise as active ingredient an effective dose of at least one compound of the formula Ia and/or Ib and/or a physiologically tolerated salt thereof in addition to conventional pharmaceutically acceptable carriers and excipients and, where appropriate, also one or more other pharmacological active substances. The pharmaceutical preparations normally comprise from 0.1 to 90% by weight of compounds of the formulae Ia and/or Ib and/or their physiologically tolerated salts.

The pharmaceutical preparations can be produced in a manner known per se. For this purpose, the active substances and/or their physiologically tolerated salts can be converted together with one or more solid or liquid pharmaceutical carriers and/or excipients into a suitable presentation or dosage form which can then be used as medicament in human medicine or veterinary medicine.

Medicaments comprising the compounds of the invention of the formulae Ia and/or Ib and/or their physiologically tolerated salts can be administered for example orally, parenterally, intravenously, rectally, by inhalation or topically, with the preferred administration being dependent on the individual case.

The skilled worker is familiar on the basis of his expert knowledge with which excipients are suitable for the desired pharmaceutical formulation.

Besides solvents, gel formers, suppository bases, tablet excipients and other active substance carriers it is possible to use for example antioxidants, dispersants, emulsifiers, antifoams, masking flavors, preservatives, solubilizers, agents for achieving a depot effect, buffer substances or colorants.

For a form for oral use, the active compounds are mixed with the additives suitable for this purpose, such as carriers, stabilizers or inert diluents, and converted by conventional methods into suitable presentations such as tablets, coated tablets, two-piece capsules, aqueous, alcoholic or oily solutions. Examples of inert carriers which can be used are gum arabic, magnesia, magnesium carbonate, potassium phosphate, lactose, glucose or starch, especially corn starch. Preparation can take place both as dry and as wet granules. Suitable as oily carriers or as solvents are, for example, vegetable or animal oils such as sunflower oil or fish liver oil. Suitable solvents for aqueous or alcoholic solutions are, for example, water, ethanol or sugar solutions or mixtures thereof. Examples of further excipients, also for other administration forms, are polyethylene glycols and polypropylene glycols.

For subcutaneous, intramuscular or intravenous administration, the active compounds are converted if desired with the substances usual for this purpose, such as solubilizers, emulsifiers or further excipients, into a solution, suspension or emulsion. Examples of suitable solvents are water, physiological saline or alcohols, for example ethanol, propanol, glycerol, as well as sugar solutions such as glucose or mannitol solutions, or else mixtures of the various solvents mentioned.

Suitable as pharmaceutical formulation for administration in the form of aerosols or sprays are, for example, solutions, suspensions or emulsions of the active substances or their physiologically tolerated salts in a pharmaceutically acceptable solvent, such as in particular ethanol or water, or a mixture of such solvents. The formulation may if required also comprise other pharmaceutical excipients such as surfactants, emulsifiers and stabilizers, and a propellant gas. Such a preparation comprises the active substance normally in a concentration of about 0.1 to 10, in particular of about 0.3 to 3 percent by weight.

The dosage of the active compounds to be administered according to the invention, or of the physiologically tolerated salts thereof depends on the individual case and should be adapted to the circumstances of the individual case as usual for an optimal effect. Thus, it naturally depends on the frequency of administration and on the potency and duration of action of the particular compounds employed for therapy or prophylaxis, but also on the type and severity of the disease to be treated, and on the gender, age, weight and individual response of the human or animal to be treated, and on whether therapy is acute or chronic or prophylaxis is intended. The dosage of the Kv1.5 blocker of the formulae Ia and/or Ib may normally vary in the range from 1 mg to 1 g per day and per person (with a body weight of about 75 kg), preferably from 5 to 750 mg per day and person. However, higher doses may also be appropriate. The daily dose of the active substance can be administered all at once or it can be divided into a plurality of administrations, for example two, three or four.

### EXPERIMENTAL SECTION

List of Abbreviations

DMAP 4-dimethylaminopyridine
EDAC N-ethyl-N′-(3-dimethylaminopropyl)carbodiimide hydrochloride
HOBT 1-hydroxy-1H-benzotriazole
RT room temperature
THF tetrahydrofuran

### Example 1

### N-(2-pyridin-3-ylethyl )-2′-{[2-(4-methoxyphenyl)acetylamino]-methyl}biphenyl-2-carboxamide

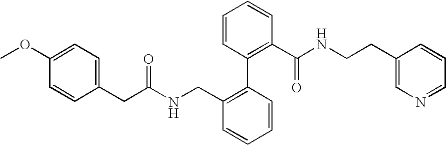

15.5 g (0.115 mol) of HOBT and 21.9 g (0.115 mol) of EDAC were added to a solution of 37.8 g (0.11 mol) of 2′-(tert-butoxycarbonylaminomethyl)-biphenyl-2-carboxylic acid (Brandmeier, V.; Sauer, W. H. B.; Feigel, M.; Helv. Chim. Acta 1994, 77(1), 70-85) in 550 ml of THF, and the reaction mixture was stirred at room temperature for 45 min. Then 14.0 g (0.115 mol) of 3-(2-aminoethyl)pyridine were added, and the mixture was stirred at RT overnight. After addition of 400 ml of water and 500 ml of ethyl acetate and vigorous stirring, the phases were separated. The organic phase was washed once with 400 ml of saturated sodium chloride solution and twice with 400 ml of saturated sodium bicarbonate solution each time. Drying over magnesium sulfate in the presence of activated carbon was followed by filtration and concentration in a rotary evaporator.

The resulting intermediate (40.7 g) was dissolved in 600 ml of methylene chloride, and then 100 ml of trifluoroacetic acid were slowly added dropwise. After stirring overnight, the reaction mixture was concentrated in vacuo. The residue was mixed with 250 ml of ethyl acetate and again concentrated in order to distill out excess trifluoroacetic acid. The resulting crude product was dissolved in 170 ml of methylene chloride, and 72.8 ml (530 mmol) of triethylamine were added dropwise, and 1 g of DMAP was added. Then, at 5-10° C., 18.7 g (100 mmol) of 4-methoxyphenylacetyl chloride were added dropwise over the course of 30 min, and the mixture was stirred at room temperature overnight. After addition of 150 ml of water and vigorous stirring, the phases were separated and the organic phase was washed once with 100 ml of sodium chloride solution, once with 25 ml of 1M hydrochloric acid and twice with 100 ml of saturated sodium bicarbonate solution each time. Drying over magnesium sulfate and activated carbon was followed by concentration in vacuo. The resulting oil was dissolved in hot acetonitrile and left to crystallize out slowly.

21.5 g of N-(2-pyridin-3-ylethyl)-2′-{[2-(4-methoxyphenyl)acetylamino]-methyl}biphenyl-2-carboxamide, melting point 116° C., were obtained.

### Example 2

### N-(2-(2-pyridyl)ethyl)-2′-(benzyloxycarbonylaminomethyl)bi-phenyl-2-carboxamide

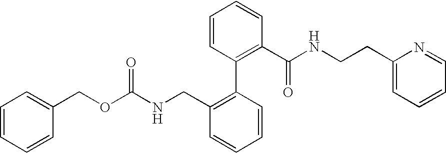

The compound was obtained by the synthetic methods indicated in WO 0125189.

### Example 3

### N-(2,4-difluorobenzyl)-2′-{[2-(4-methoxyphenyl)acetylamino]-methyl}biphenyl-2-carboxamide

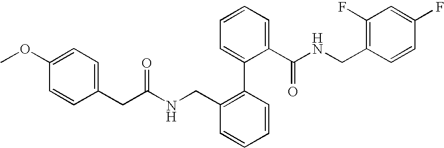

The compound was obtained by the synthetic method indicated in WO 0125189.

### Example 4

### N-(2-(2-pyridyl)ethyl)-(S)-2′-(α-methylbenzyloxycarbonyl-aminomethyl)biphenyl-2-carboxamide

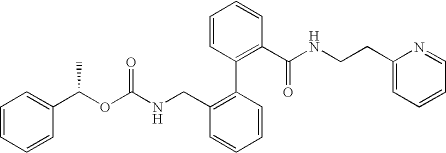

The compound was obtained by the synthetic method indicated in WO 0125189.

### Example 5

### 2-(Butyl-1-sulfonylamino)-N-[1(R)-(6-methoxypyridin-3-yl)-propyl]benzamide

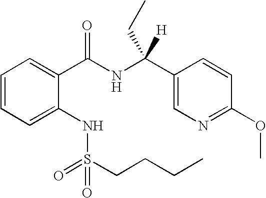

### a) 2-(Butyl-1-sulfonylamino)benzoic acid

20 g (188 mmol) of sodium carbonate were added to a suspension of 20 g (146 mmol) of 2-aminobenzoic acid in 250 ml of water. Then 11.4 g (72.8 mmol) of butylsulfonyl chloride were added dropwise, and the reaction mixture was stirred at room temperature for 2 days. It was acidified with concentrated hydrochloric acid and stirred at room temperature for 3 hours, and the precipitated product was filtered off with suction. Drying in vacuo resulted in 9.6 g of 2-(butyl-1-sulfonylamino)benzoic acid.

### b) 1-(6-Methoxypyridin-3-yl)propylamine

3 ml (23.2 mmol) of 5-bromo-2-methoxypyridine were added to a solution of 10.2 ml of butyllithium (2.5 M solution in hexane; 25.5 mmol) in 50 ml of diethyl ether at −70° C. After 10 min, 1.4 ml (19.5 mmol) of propionitrile were added. After 2 hours at −70° C., the reaction mixture was slowly allowed to reach room temperature. Then 2.2 g of sodium sulfate decahydrate were added, and the mixture was left to stir for 1 hour. After subsequent addition of 5 g of magnesium sulfate and after brief stirring, the salts were filtered off and the filtrate was concentrated. The residue was dissolved in 70 ml of methanol and, at 0° C., 1.1 g (28 mmol) of sodium borohydride were added. After stirring overnight, the reaction mixture was adjusted to pH 2 with concentrated hydrochloric acid and concentrated in a rotary evaporator. The residue was mixed with 10 ml of water and extracted once with diethyl ether. The aqueous phase was then saturated with sodium bicarbonate and concentrated in vacuo, and the residue was extracted with ethyl acetate. Drying and concentration of the ethyl acetate extracts resulted in 1.4 g of racemic 1-(6-methoxypyridin-3-yl)propylamine.

### c) 2-(Butyl-1-sulfonylamino)-N-[1(R)-(6-methoxypyridin-3-yl)propyl]-benzamide

4.4 g (32.7 mmol) of 1-hydroxy-1H-benzotriazole and 6.3 g (32.7 mmol) of N-ethyl-N′-(3-dimethylaminopropyl)carbodiimide hydrochloride were added to a solution of 8.0 g (31.1 mmol) of 2-(butyl-1-sulfonylamino)benzoic acid in 250 ml of tetrahydrofuran, and the reaction mixture was stirred for 90 min. Then a solution of 5.4 g (32.7 mmol) of racemic 1-(6-methoxy-pyridin-3-yl)propylamine in 20 ml of tetrahydrofuran was added dropwise, and the mixture was stirred overnight. The reaction mixture was mixed with 250 ml of water and extracted with 300 ml of ethyl acetate. The organic phase was extracted 5 times with 100 ml of saturated sodium bicarbonate solution each time and then dried over magnesium sulfate. 9.0 g of 2-(butyl-1-sulfonylamino)-N-[1-(6-methoxypyridin-3-yl)propyl]benzamide were obtained.

Separation of the enantiomers took place by preparative HPLC on a Chiralpak ADH column (250×4.6 mm); eluent: heptane/ethanol/methanol 10:1:1; temperature: 30° C.; flow rate: 1 ml/min. The first to be eluted at a retention time of 5.9 min was 4.0 g of 2-(butyl-1-sulfonylamino)-N-[1(R)-(6-methoxypyridin-3-yl)propyl]benzamide. A mixed fraction was followed by, at a retention time of 7.2 min, 3.0 g of 2-(butyl-1-sulfonylamino)-N-[1(S)-(6-methoxypyridin-3-yl )propyl]benzamide.

2 g of 2-(butyl-1-sulfonylamino)-N-[1(R)-(6-methoxypyridin-3-yl)propyl]-benzamide were dissolved in 9 ml of hot isopropanol and then 8 ml of hot water were added, and the reaction mixture was allowed to cool slowly overnight. After filtration with suction at 0° C., 1.5 g of 2-(butyl-1-sulfonylamino)-N-[1(R)-(6-methoxypyridin-3-yl)propyl]benzamide were obtained as colorless crystalline needles; melting point 97° C.

### Example 6

### 2-(Butyl-1-sulfonylamino)-N-(cyclopropylpyridin-3-ylmethyl)-5-methylbenzamide

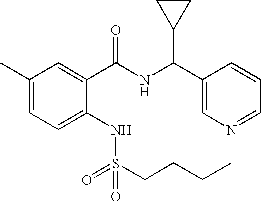

The compound was obtained by the synthetic method indicated in WO 02088073.

### Example 7

### (S)-5-Fluoro-2-(quinoline-8-sulfonylamino)-N-(1-phenylpropyl)-benzamide

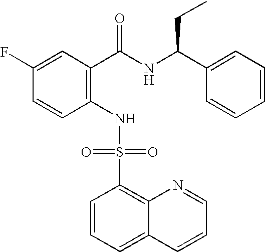

### a) 5-Fluoro-2-(quinoline-8-sulfonylamino)benzoic acid

A reaction mixture of 10.0 g (64 mmol) of 5-fluoro-2-aminobenzoic acid, 16.3 g (193 mmol) of sodium bicarbonate and 16.3 g of 8-quinolinesulfonyl chloride in 325 ml of water and 325 ml of ethyl acetate was stirred at RT overnight. The aqueous phase was separated off and extracted once with 50 ml of ethyl acetate. The aqueous phase was then acidified with conc. hydrochloric acid and stirred for 2 h. The precipitate which separated out was filtered off with suction and dried in vacuo to result in 19.5 g of 5-fluoro-2-(quinoline-8-sulfonylamino)benzoic acid.

### b) 5-Fluoro-2-(quinoline-8-sulfonylamino)-N-(1-phenylpropyl)benzamide

5.7 g of the title compound were obtained from 5.5 g (15.9 mmol) of 5-fluoro-2-(quinoline-8-sulfonylamino)benzoic acid and 2.3 g (16.7 mmol) of (S)-phenylpropylamine by the method in WO 02100825.

M.p.: 163° C.

### Example 8

### (S)-5-Fluoro-2-(quinoline-8-sulfonylamino)-N-(1-phenylpropyl)-benzamide Sodium Salts

2 ml of a 30 percent strength sodium methanolate solution were added to a solution of 5 g of the compound of example 7 in 120 ml of ethyl acetate. The precipitated sodium salt was filtered off with suction and recrystallized from 25 ml of ethanol to result in 3.3 g of the title compound.

Pharmacological Investigations

Determination of the Activity on the Kv1.5 Channel

Human Kv1.5 channels were expressed in xenopus oocytes. For this purpose, firstly oocytes were isolated from Xenopus laevis and defolliculated. Kv1.5-encoding RNA synthesized in vitro was then injected into these oocytes. After Kv1.5 protein expression for 1-7 days, Kv1.5 currents were measured on the oocytes using the two-microelectrode voltage clamp technique. The Kv1.5 channels were in this case usually activated with voltage jumps lasting 500 ms to 0 mV and 40 mV. A solution of the following composition flowed through the bath: NaCl 96 mM, KCl 2 mM, CaCl₂ 1.8 mM, MgCl₂ 1 mM, HEPES 5 mM (titrated to pH 7.4 with NaOH). These experiments were carried out at room temperature. The following were employed for data acquisition and analysis: Geneclamp amplifier (Axon Instruments, Foster City, USA) and MacLab D/A converter and software (ADInstruments, Castle Hill, Australia). The substances of the invention were tested by adding them in various concentrations to the bath solution. The effects of the substances were calculated as percent inhibition of the Kv1.5 control current which was obtained when no substance was added to the solution. The data were then extrapolated using the Hill equation in order to determine the inhibitory concentrations IC₅₀ for the respective substances.

The IC₅₀ values determined in this way for the compounds listed below were as follows:

| Example No. | IC₅₀ [μM] |
|---|---|
| 1 | 4.7 |
| 2 | 0.7 |
| 3 | 1.4 |
| 4 | 0.2 |
| 5 | 10 |
| 6 | 1.0 |
| 7 | 1.1 |

The direct effect of Kv1.5 blockers on the contractility of the pig left atrium is shown below (A). In the second design of experiment (B), the effect of the improved atrial contractility on impeded ventricular filling (diastolic dysfunction) is demonstrated.

A) Test of the Effect of Kv1.5 Blockers on Atrial Contractility in Anesthetized Pigs

Material and methods: German Landrace pigs were premedicated by an intramuscular injection of 2.5-3.5 mg/kg each of xylazine, tiletamine and zolazepam in a mixing syringe. The anesthesia was induced with pentobarbital (approx. 30 mg/kg i.v.) and maintained by continuous infusion of pentobarbital (12-17 mg/kg/h).

After induction of anesthesia and after a tracheotomy, the animals were intubated and ventilated with a mixture of ambient air with 40% oxygen.

In a further series of experiments, the test substance was administered only after one hour of atrial fibrillation induced by persistent high-frequency stimulation (1200 beats/min) of the right atrium. In this case, the parameters of atrial contractility were recorded before/after the period of fibrillation and after administration of the test substance and compared with those after the vehicle control.

The compound of example 1 leads to a statistically significant improvement in atrial function in pigs both in normal sinus rhythm (table 1) and after one hour of atrial fibrillation (table 2). The improved atrial function was equally evident from both parameters, the LASS index and the LACC steepness. The effect of compound of example 1 after one hour of atrial fibrillation should be particularly emphasized because the contractility has fallen to a level of 57-69% of the initial value there. In this situation, compound of example 1 was able to improve the contractility to above the base line level (before atrial fibrillation).

The compound of example 1 improves atrial contractility considerably both in sinus rhythm and after atrial fibrillation, where the atrial contractility is decreased to a pathophysiologically significant extent by the process called electrical remodeling.

TABLE 1
Effect of the compound of example 1, 1 mg/kg i.v., on parameters
of atrial contractility in sinus rhythm.

| | LASS index | | LACC steepness (cm/s) | |
|---|---|---|---|---|
| | Before | After | Before | After |
| | administration | administration | administration | administration |
| | of example 1 | of example 1 | of example 1 | of example 1 |
| Absolute value | 0.159 ± 0.021 | 0.206 ± 0.017** | −0.110 ± 0.016 | −0.152 ± 0.02** |
| Increase in % | | +29.5% | | +38.2% |

*p < 0.05;
**p < 0.01

The ventilation rate and volume were governed by regularly measured blood gas and pH levels. The body temperature was continuously recorded and controlled by a heated underlay and/or red light lamp and/or warming of the breathed air to keep it constant (about 37-38° C.).

The following blood vessels of the pigs were exposed and cannulated:

External jugular vein (anesthesia infusion), carotid artery (introduction of a tip manometer catheter in the left ventricle to record the pressure there), lateral saphenous vein (dosage of test substance), epigastrica cranialis superficialis vein (fluid infusion), femoral artery (peripheral blood pressure recording) and femoral vein (introduction of MAP catheter right atrium).

The parameters responsible for the contractility are determined using two ultrasonic sensors on the left atrium (P/N JP 5-2, Triton Technology®) [references 1 and 2]. These two piezoelectric sensors are implanted in the cranio-caudal direction through punctuate incisions at the extreme edge of the atrium. The incisions were each closed with a U stitch (2-0 Vicryl®).

The two ultrasonic sensors were than connected to the evaluation unit. In addition, a pressure-measuring catheter was implanted at the ventral edge of the atrium in order to record the left atrial pressure.

The left atrial systolic shortening (LASS) was determined from the atrial diameter at the start of the active atrial pressure curve and the minimum diameter. Since the atrial contractility depends on the initial extension, the left atrial systolic shortening was divided by the value at the start of active atrial contraction (LASS index). The maximum steepness of the contraction curve was determined by importing the raw data points to Microsoft Excel for calculation of the maximum steepness of the curve. In order to exclude breathing-related variations, at least 10 cardiac cycles were analyzed. This parameter, which likewise indicates an improved contractility, is referred to as LASS steepness.

Recording of the initial values was followed by infusion for 10 minutes of the vehicle which was used later and is composed of 0.5 ml of DMSO, 2.5 ml of PEG and 2.0 ml of glucose G20. Subsequently, in sinus rhythm, 1 mg/kg of the test substance dissolved in the abovementioned vehicle was administered intravenously (i.v.).

TABLE 2

| | LASS index | | | LACC steepness (cm/s) | | |
|---|---|---|---|---|---|---|
| | Before | After | Exam- | Before | After | Example |
| | AF | AF | ple 1 | AF | AF | 1 |
| Abso- | 0.174 ± | 0.118 ± | 0.203 ± | −0.175 ± | −0.099 ± | −0.197 ± |
| lute val- | 0.020 | 0.025 | 0.023** | 0.020 | 0.023 | 0.028** |
| ues | | | | | | |
| In % of | 100% | 69.4% | 116% | 100% | 56.6% | 112.6% |
| the init | | | | | | |
| ial val- | | | | | | |
| ue | | | | | | |

Effect of the compound of example 1, 1 mg/kg i.v., on parameters of atrial contractility after 1 hour atrial fibfrillation (AF).
*p < 0.05;
**p < 0.01
Example 5 likewise sshowed an improved left-atrial contractility in the same experimental design in sinus rhythm.
The atrial contractility (LASS) was improved by 68% in sinus rhythm after 1 mg/kg i.v. (table 3).

TABLE 3
Effect of the compound of example 5, 1 mg/kg i.v., on parameters
of atrial contractility in sinus rhythm.

LASS

| | Before administration | After administration |
|---|---|---|
| | of example 5 | of example 5 |
| Absolute values | 4.91 ± 0.63 | 8.25 ± 1.43 |
| Increase in % | | 68% |

*p < 0.05; n = 8.

B) Improved Left Ventricular Ejection after Kv1.5 Blockers in a Model of Diastolic Dysfunction

Methods: Pigs were anesthetized as described in A) and thoracotomized. A flow sensor was attached over the aorta to measure the cardiac output. In a stable hemodynamic situation, air (approx. 30 ml) was instilled into the pericardial using a cannula. The intention of pericardial air instillation is to impede ventricular filling, which eventually leads to a decreased cardiac output (diastolic heart failure). The aim of the experiment was to demonstrate that the reduced cardiac output can be increased by enhanced atrial contractility through the atrial contractility which is increased by Kv1.5 blockade bringing about an improvement in the impeded ventricular filling.

The pericardial air instillation leads to a distinct reduction in the cardiac output (table 3). It was possible considerably to increase the latter by administration of the compound of example 1 (3 mg/kg), infused in 30 min (n=11). The maximum increase in the reduced cardial output by the compound of example 1 was 25%.

Kv1.5 blockade by the compound of example 1 increases the cardiac output when ventricular filling is impeded. The results show that Kv1.5 blockade is particularly effective for diastolic heart failure.

TABLE 3
Cardiac output (l/min) in pigs (n = 11) with pericardial air instillation
to impede ventricular filling (diastolic dysfunction or heart failure) before
and after administration of the compound of example 1 (3 mg/kg) i.v.
over 30 min.

| | | | % of the initial | % of the initial |
|---|---|---|---|---|
| | Average | | value before | value before |
| | (l/min) | SEM | instillation | example 1 |
| Baseline value before | 2.14 | 0.16 | 100% | |
| pericardial filling | | | | |
| Baseline value after | 1.68 | 0.13 | 78% | |
| pericardial filling | | | | |
| Vehicle 5′ | 1.60 | 0.08 | 80% | |
| 10′ | 1.57 | 0.09 | 78% | |
| 15′ | 1.55 | 0.09 | 77% | |
| 20′ | 1.53 | 0.09 | 76% | |
| 25′ | 1.54 | 0.09 | 77% | |
| 30′ | 1.49 | 0.09 | 74% | |
| Example 1 | 1.53 | 0.14 | 71% | 100% |
| 2′ | 1.55 | 0.15 | 72% | 102% |
| 5′ | 1.58 | 0.16 | 73% | 104% |
| 10′ | 1.69 | 0.19 | 78% | 111% |
| 15′ | 1.78 | 0.21 | 82% | 117% |
| 20′ | 1.84 | 0.22 | 84% | 121% |
| 25′ | 1.88 | 0.21 | 86% | 123% |
| 30′ | 1.90 | 0.21 | 87% | 125% |
| 35′ | 1.83 | 0.19 | 85% | 120% |
| 40′ | 1.79 | 0.17 | 83% | 117% |
| 50′ | 1.76 | 0.18 | 82% | 116% |
| 60′ | 1.69 | 0.16 | 79% | 111% |
| 70′ | 1.65 | 0.15 | 78% | 108% |

### REFERENCES

Leistad E, Aksnes G, Verburg E, Christensen G. Atrial contractile dysfunction after short-term atrial fibrillation is reduced by verapamil but increased by BAY K8644. Circulation 1996;93:1747-1754.

Recordati G, Lombardi F, Malliani A, Brown A M. Instantaneous dimensional changes of the right atrium of the cat. J Appl Physiol 1974;36:686-692.

What is claimed is:

1. A method for the treatment or prophylaxis of heart failure comprising adminstering to a patient in need thereof an effective amount of a compound of the formula Ia or Ib 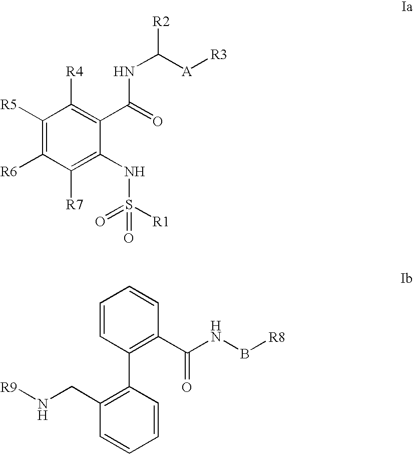
or a physiologically tolerated salt thereof,
wherein the meanings are
R1 is alkyl having 3, 4 or 5 C atoms or quinolinyl,
R2 is alkyl having 1, 2, 3 or 4 C atoms or cyclopropyl;
R3 is phenyl or pyridyl,
where the phenyl or pyridyl is unsubstituted or substituted by 1 or 2 substituents selected from the group consisting of F, Cl, CF₃, OCF₃, alkyl having 1, 2 or 3 carbon atoms and alkoxy having 1, 2 or 3 carbon atoms;
A is —CₙH₂ₙ—;
n is 0, 1 or 2;
R4, R5, R6 and R7
are independently of one another hydrogen, F, Cl, CF₃, OCF₃, CN, alkyl having 1, 2 or 3 carbon atoms, alkoxy having 1, 2 or 3 carbon atoms;
B is —CₘH₂ₘ—;
m is 1 or 2;
R8 is alkyl having 2 or 3 carbon atoms, phenyl or pyridyl, where the phenyl or pyridyl is unsubstituted or substituted by 1 or 2 substituents selected from the group consisting of F, Cl, CF₃,
OCF₃, alkyl having 1, 2 or 3 carbon atoms and alkoxy having 1, 2 or 3 C atoms;
R9 is C(O)OR10 or COR10;
R10 is —CₓH₂ₓ—R11;
x is 0, 1 or 2;
R11 is phenyl,
where the phenyl is unsubstituted or substituted by 1 or 2 substituents selected from the group consisting of F, Cl, CF₃,
OCF₃, alkyl having 1, 2 or 3 carbon atoms and alkoxy having 1, 2 or 3 carbon atoms.

2. The method of claim 1 wherein the compound is selected from the group consisting of
N-(2-pyridin-3-ylethyl)-2′-{[2-(4-methoxyphenyl)acetylamino]methyl}bi-phenyl-2-carboxamide,
N-(2-(2-pyridyl)ethyl)-2′-(benzyloxycarbonylaminomethyl)biphenyl-2-carboxamide,
N-(2,4-difluorobenzyl)-2′-{[2-(4-methoxyphenyl)acetylamino]methyl}-biphenyl-2-carboxamide,
N-(2-(2-pyridyl)ethyl)-(S)-2′-(α-methylbenzyloxycarbonylaminomethyl)bi-phenyl-2-carboxamide,
2-(butyl-1-sulfonylamino)-N-[1(R)-(6-methoxypyridin-3-yl)propyl]benzamide,
2-(butyl-1-sulfonylamino)-N-(cyclopropylpyridin-3-ylmethyl)-5-methyl-benzamide,
(S)-5-fluoro-2-(quinoline-8-sulfonylamino)-N-(1-phenylpropyl)benzamide, and the physiologically tolerated salts thereof.

3. The method of claim 1 wherein the heart failure is diastolic heart failure.

4. The method of claim 2 wherein the heart failure is diastolic heart failure
